(19) United States
(12) Patent Application Publication
(10) Pub. No.: US 2019/0001082 A1
(43) Pub. Date: Jan. 3, 2019

(54) WEARABLE AEROSOL INHALER DEVICE AND METHOD

(71) Applicants: Elizabeth Tarangelo, Encinitas, CA (US); Kayla Simon, Jackson, NJ (US)

(72) Inventors: Elizabeth Tarangelo, Encinitas, CA (US); Kayla Simon, Jackson, NJ (US)

(21) Appl. No.: 16/023,566

(22) Filed: Jun. 29, 2018

Related U.S. Application Data
(60) Provisional application No. 62/526,629, filed on Jun. 29, 2017.

(51) Int. Cl. A61M 15/00

(52) U.S. Cl. CPC A61M 15/009; A61M 15/0021; A61M 2209/088; A61M 15/0025; A61M 15/0066; A61M 15/0068

(57) ABSTRACT

A wearable inhaler includes a wristband closed by at least one separation joint. The wristband is flexible or hinged to open between a first end and a second end at the at least one separation joint. An operable inhaler is disposed within the first end. In an open configuration, a partially exposed section and a mouth piece are exposed to provide a metered dose of aerosol to a user. A method of use of a wristband inhaler is also described.

### CROSS-REFERENCE TO RELATED APPLICATIONS

This application claims priority to and the benefit of co-pending U.S. provisional patent application Ser. No. 62/526,629, WEARABLE AEROSOL INHALER, filed Jun. 29, 2017, which application is incorporated herein by reference in its entirety.

### FIELD OF THE APPLICATION

The application relates to an inhaler and particularly to a wearable inhaler.

### BACKGROUND

A standard asthma inhaler is a MDI (metered-dose inhaler). The standard asthma inhaler typically includes the following parts: a metered-dose valve, an internal canister housing the metered-dose valve and dosage in the hundreds, an actuator, and a nozzle to direct the aerosol spray. These parts collectively allow for the release of medication in the form of aerosol droplets from a nozzle when a user applies pressure to the top of the canister.

### SUMMARY

According to one aspect, a wearable inhaler includes a wristband closed by at least one separation joint. The wristband is flexible or hinged to open between a first end and a second end at the at least one separation joint. An operable inhaler is disposed within the first end. In an open configuration, a partially exposed section and a mouth piece are exposed to provide a metered dose of aerosol to a user.

In one embodiment, a longitudinal axis of the operable inhaler is aligned about axially or co-axially within the wristband.

In another embodiment, the mouth piece includes at least one bite pad.

In yet another embodiment, the second end provides a protective cover for the operable inhaler disposed within the first end.

In yet another embodiment, the operable inhaler includes a single dose inhaler.

In yet another embodiment, the operable inhaler includes a multiple dose inhaler.

In yet another embodiment, the operable inhaler includes a re-fillable inhaler.

In yet another embodiment, the operable inhaler includes a trap refill door.

In yet another embodiment, the operable inhaler includes a filling valve.

In yet another embodiment, the operable inhaler includes an actuator slidably coupled to a housing shell.

In yet another embodiment, the operable inhaler includes a backboard about flush against a back of an outer casing.

In yet another embodiment, the actuator is spring biased away from the backboard.

In yet another embodiment, the operable inhaler includes a metered dose valve.

In yet another embodiment, the operable inhaler includes a metering chamber to fill a predetermined dosage.

In yet another embodiment, the operable inhaler includes a retaining cup to hold an on-deck dose.

In yet another embodiment, the operable inhaler includes a dose counter.

In yet another embodiment, the operable inhaler includes a dose button disposed in a backboard.

In yet another embodiment, the operable inhaler includes an adjustable strap disposed between the first end and the second end.

In yet another embodiment, the wristband includes a second separation point at one or more clasps or pins of the adjustable strap.

In yet another embodiment, the operable inhaler includes a disposable or a replaceable pod.

In yet another embodiment, the wearable inhaler further includes a watch face or a fitness tracker to provide a physiological monitoring.

According to another aspect, a method of use of a wristband inhaler includes: providing a wristband closed by at least one separation joint, the wristband flexible or hinged to open between a first end and a second end at the at least one separation joint, and an operable inhaler disposed within the first end, wherein in an open configuration, a partially exposed section and a mouthpiece are exposed to provide a dose of aerosol to a user; opening the wristband inhaler by pulling apart the first end and the second end; placing a user's lips about the partially exposed section; biting on the mouthpiece by the user; and pushing forward to release a metered dose of an aerosol to the user.

The foregoing and other aspects, features, and advantages of the application will become more apparent from the following description and from the claims.

### BRIEF DESCRIPTION OF THE DRAWINGS

The features of the application can be better understood with reference to the drawings described below, and the claims. The drawings are not necessarily to scale, emphasis instead generally being placed upon illustrating the principles described herein. In the drawings, like numerals are used to indicate like parts throughout the various views.

FIG. 1 shows a side view of an exemplary wristband inhaler device according to the Application in a closed position;

FIG. 2 shows an isometric view of the wristband inhaler device of FIG. 1 in an open position when not yet triggered;

FIG. 3 shows a side view of the device of FIG. 1 in the opened position;

FIG. 4 is a drawing which illustrates the actuator and the bite/push method;

FIG. 5 shows an isometric partial view of another embodiment of an exemplary wristband inhaler device according to the Application;

FIG. 6 shows a drawing which illustrates an exemplary spring and dispensing assembly;

FIG. 7A shows a drawing of a side view of another exemplary embodiment of a wristband inhaler device;

FIG. 7B shows an isometric view of a first end of the wristband inhaler device of FIG. 7A;

FIG. 7C shows an isometric view of the wristband inhaler device of FIG. 7A;

FIG. 8A shows a drawing of a side view of yet another exemplary embodiment of a wristband inhaler device with an adjustable strap;

FIG. 8B shows a drawing an isometric view of a first end of the wristband inhaler device of FIG. 8A;

FIG. 8C shows a drawing an isometric view of the wristband inhaler device of FIG. 8A;

FIG. 9A shows a drawing of top view another exemplary first end;

FIG. 9B shows a drawing of an isometric view of the first end of FIG. 9A;

FIG. 9C shows a drawing of a side view of the first end of FIG. 9A;

FIG. 10A shows a drawing of an exemplary mechanical dose counter of the prior art; and

FIG. 10B shows a drawing of an exemplary dose dispensing device of the prior art.

### DETAILED DESCRIPTION

As described hereinabove, a standard asthma inhaler is a MDI (metered-dose inhaler). The standard asthma inhaler typically includes the following parts: a metered-dose valve, an internal canister housing the metered-dose valve and dosage in the hundreds, an actuator, and a nozzle to direct the aerosol spray. These parts collectively allow for the release of medication in the form of aerosol droplets from a nozzle when a user applies pressure to the top of the canister.

The standard asthma inhaler is the most common device and method for asthma symptom relief and standard practice for asthma patients, as well as various other respiratory diseases. However, there are multiple flaws with this method. The inhaler itself employs a design that requires multiple complex steps/actions which results in hand-to-mouth coordination issues. Operation problems related to the hand-to-mouth coordination often lead to a large portion of the dosage of Albuterol going unused. Another issue is the bulkiness of the inhaler, which leads asthma patients to leave their inhaler behind when they are going about daily activities. Thus, asthma patients often do not have their medication with them when they may need it the most.

What is needed is an inhaler that is both less complicated to trigger and easy for a user to carry with them at all times, via a body-mounted device.

Beginning with two exemplary inhalers of the prior art, U.S. Pat. No. 5,590,645, Inhalation device, issued Jan. 7, 1997 to Davies, et al. describes an inhalation device by means of which a user can inhale medicament in the form of a powder. The '645 patent teaches a standard inhalation device that features the components of a typical inhaler, which illustrate the technical and aesthetic flaws of prior art inhalers. The relatively large device is impractical in emergencies and on-the-go situations. Technical flaws include the hand-to-mouth coordination problem and a lack of a replaceable canister.

U.S. Pat. No. 9,550,032, Wearable wrist inhaler, issued Jan. 24, 2017 to Baldwin, describes wearable asthma inhaler configured in the form of a wristwatch to provide fast effective relief of asthma symptoms. The '032 patent describes a wrist-mounted inhaler in two embodiments; one referring to a dry powder inhaler, and the other an aerosol. The aerosol bracelet inhaler is a three-piece structure that detaches to provide access to an inhaler with a standard canister that is not refillable, but also continues to use the same method of pushing on the back of the canister to release the medication. The user would rip off the bracelet portion of the wearable wrist inhaler, and then proceed to use the third portion in a similar way to a standard inhaler. However, a problem with the design of the inhaler of the '032 patent is that within the inhaler part of the device, several more pieces are required, and several steps are required, to actually access the medication. Contained within the device is a nozzle holder, several chambers, and removable compartments. These compartments are all held together through a friction fit. The '032 patent description makes it clear that the inhaler is only operable when released entirely from the wristband. After, release from the wristband, the device of the '032 patent is a stand-alone inhaler device, similar in structure to a traditional inhaler.

The wristband inhaler device of the Application differs from the prior art for at least one or more of the following reasons, the wristband inhaler device of the Application: 1) uses one streamlined wristband with the inhaler integrated directly into it, 2) a refillable canister of lower dosage, and 3) a closure (e.g. a magnetic closure) to hold the band together. Another difference is the use of a “bite method,” in which a user places their teeth onto bite pads integrated into the mouthpiece, then pushes forward toward the bracelet, which directly triggers the release of any suitable aerosol (typically an albuterol) from the canister. This new method results in the wristband inhaler device of the Application being bite-actuated, thus eliminating the confusion of hand-to-mouth coordination.

Typically, the wristband inhaler device of the Application is a wearable Albuterol inhaler for asthma patients who may suffer from asthma attacks while on-the-go. The device can take the form of an adjustable, waterproof wristband. The band can possess a breaking point, so that a user can detach the wristband from her person, exposing the mouthpiece for the specially-sized Albuterol dispenser contained within the band. In some embodiments, the inhaler of the Application includes one or more of the following features: a metered-dose valve, a refillable reservoir, an actuator with bite pads to dispense the medication, and a dose counter. A metered-dose valve allows the wearable wrist inhaler to maintain a correct amount of medication within the band with each pump of medication. The user clamps her teeth around the actuator and pushes forward into the band as she inhales in order to dispense one dose of Albuterol. The device can be used with suitable medicaments that can be dispensed in a liquid or aerosol form.

The wristband inhaler device of the Application relates to an inhalation device and method for dispensing medication in aerosol form, and more specifically is geared towards those users who need or desire the portability and convenience of a wearable apparatus. The device can use a pressurized canister outfitted with a dispensing valve and a moveable actuator in a wearable casing to deliver pre-determined doses of pharmaceutical agents to a user.

In some embodiments, the wristband inhaler device of the Application can provide a waterproof, wearable Albuterol inhaler that is refillable. The wristband inhaler device of the Application can serve specifically as an accessible and compact dispenser that operates with a single motion. The device provides quick and effective relief for a user experiencing asthma symptoms while on-the-go, who needs her medication within immediate reach.

In some embodiments, the wristband inhaler device includes a band which separates at a “detachment point.” The point of detachment can be bordered by magnets in order to hold the two ends securely together until pulled apart. The separation of the two ends reveals an inner cavity that contains the inhaler. This configuration and placement on the wrist makes the inhaler easily accessible to the user at a moment's notice.

The band can be constructed in a variety of shapes, colors, and appearances to fit individual lifestyles, e.g. for children, sports activities, or professional settings. As the inhaler is integrated directly into the band, the specially sized inhaler is discrete and can thus go unnoticed until the user has a need for their medication. In some embodiments, markings on the band can signify the wristband's status as a medical device in order to make bystanders aware of the user's condition in case help is needed.

In typical embodiments, the waterproof nature of the band and components makes the inhaler rugged and able to withstand many different environments, whether the user is on land or in water.

To make the internal canister refillable, for example, the raised portion of the wristband can include any suitable opening, such as, for example, a trap door or slideable piece on the underside to make the canister easy to remove and refill.

The device is bite-actuated, which allows the user to bite down on bite pads and push forward on the actuator in one seamless action. In some embodiments, there can be an angled nozzle. The user can then breathe in the medication effectively and quickly.

A dose counter is typically integrated into the device with the ability to document up to the highest numbers of doses the canister can hold. As the user causes the release of a dose, the counter will show the number of doses remaining in the device. Any suitable electronic, electro-mechanical, or mechanical dose counter can be used. Such dose counters are well known to those skilled in the art.

The new wrist inhaler of the Application delivers asthma medication more quickly and effectively than a traditional MDI inhaler, and gives users peace of mind because the medication is in an accessible and convenient location where it will not get lost. Users will not have to waste time searching for the device in any situation, which is especially important during asthma attacks because patients often become panicked. The device can make the difference in a patient's stability and give them the ability to breathe easy.

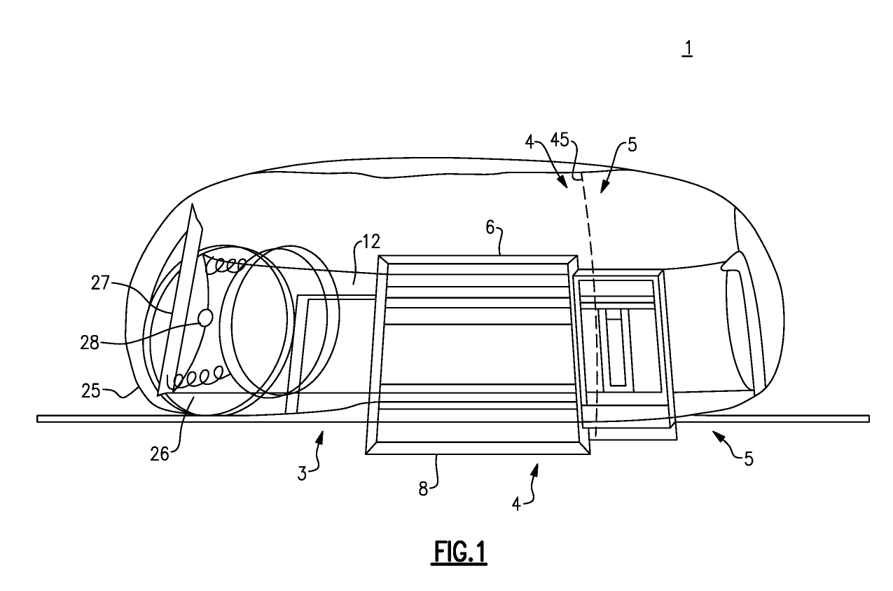

Following definitions, the wearable aerosol inhaler is described in more detail hereinbelow with reference to FIG. 1-FIG. 9C.

### Definitions

The closed position is defined as when the device is one continuous band, with the magnets sealing the inhaler inside.

The open position is defined as when magnets are separated at the breaking point, causing two ends to open and a section of the actuator to protrude from the casing with the mouthpiece exposed.

Mouthpiece refers to the section of the actuator that the user will place her teeth upon in order to trigger the release of the medicament.

Bite pads refers to the material encircling the mouthpiece on which the user will bite down.

Actuator refers to the unit consisting of the mouthpiece and internal canister.

Canister refers to the reservoir housing the medicament and the dispensing assembly.

Metered-dose valve refers to the dispensing assembly which interacts with a nozzle to release a pre-determined dosage when triggered.

Filling valve refers to a check valve or one-way valve that is stoppered by default in order to maintain the pressure within the canister, and once unplugged allows for another canister to dispense its contents one pump at a time.

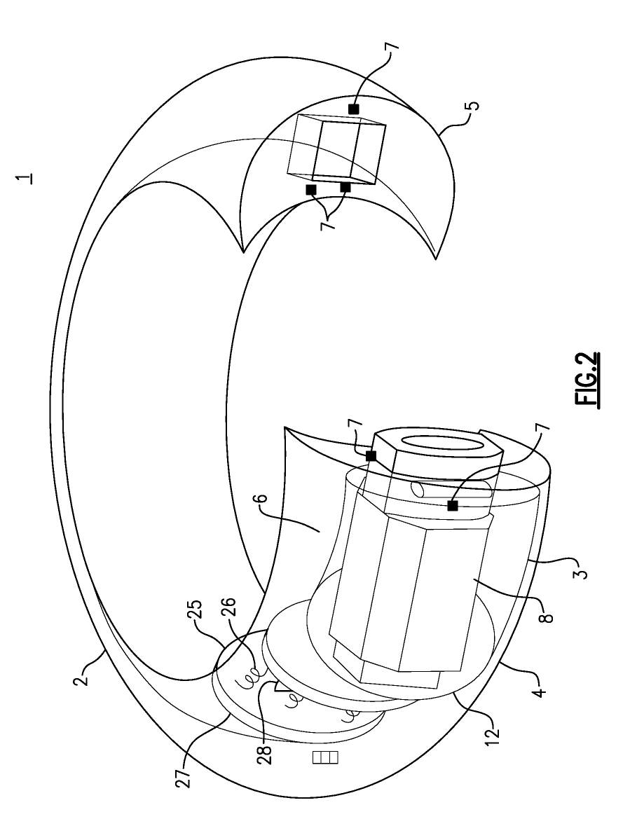

FIG. 1 shows a side view of an exemplary wearable wristband inhaler device according to the Application in a closed default position. In typical embodiments, the device can be water resistant, or more likely waterproof. Note also that in some embodiments, the wearable aerosol inhaler 1 can be an adjustable wristband 2. This exemplary wristband 2 of FIG. 1 is configured so as to wrap around a user's wrist and to conform to its size. The wristband 2 tapers to a raised portion of the band comprising of a first end 4 which contains a housing shell 6 for the internal components of the inhaler 3. A longitudinal axis of the inhaler is aligned about axially or co-axially within the wristband. A hollowed second end 5 houses a protruding section of the actuator 9 and mouthpiece 10. Magnets 7 which are embedded into both the actuator and the casing facing the actuator, line up, and form a secure seal between first end 4 and second end 5 until a user pulls them apart into the open position. Magnets 7 are shown in rough positions in FIG. 1, FIG. 2 to show concept of wristband closure. Magnets 7 can be present in any suitable number in any suitable positions at first end 4 and corresponding locations of second end 5. The wristband separates at dotted line 45 when the user pulls first end 4 and second end 4 apart into the open position. In the wristband closed configuration, the second end provides a protective cover for the operable inhaler disposed within the first end.

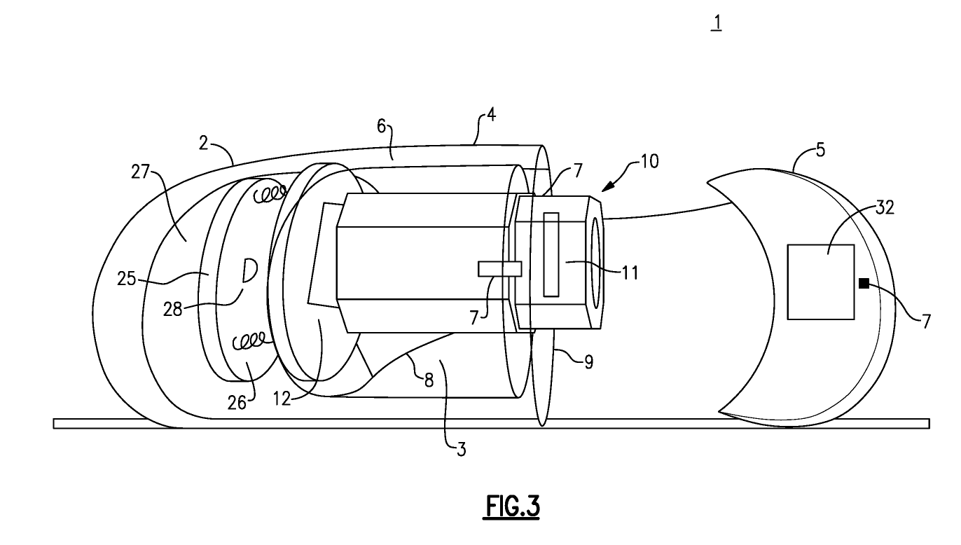

FIG. 2 and FIG. 3 illustrate the exemplary embodiment of the wearable aerosol inhaler 1 of FIG. 1, in an open position prior to use. The actuator 8 sits primarily in housing shell 6 within first end 4. A partially exposed section 9 narrows slightly to mouthpiece 10, over which the user will close their lips around. On the top and bottom of the mouthpiece are bite pads 11 covering the surface. The user will place their teeth on these in order to gain a strong grip on mouthpiece 10. Bite pads 11 can include one or more ridges or troughs, or any combination thereof, to enhance the contact between a user's teeth and mouthpiece 10.

Within the actuator 8 is the internal canister 12 that serves as a reservoir for the medicament. In the embodiment of FIG. 1, the internal canister 12 sits on and slides along tracks 13 which secure its orientation. The canister 12 can be of a compact size so as to hold a smaller amount of dosage than traditional canisters, making the medicament accessible for immediate need. A cavity 32 in the second end can accept any protruding part of the inhaler of the first end, such as at least part of mouthpiece 10, for protection when the wristband is closed.

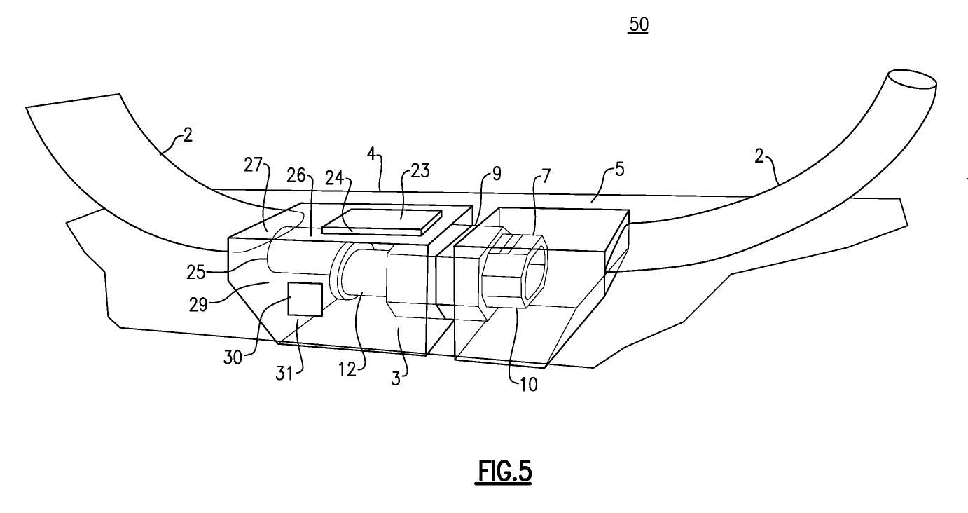

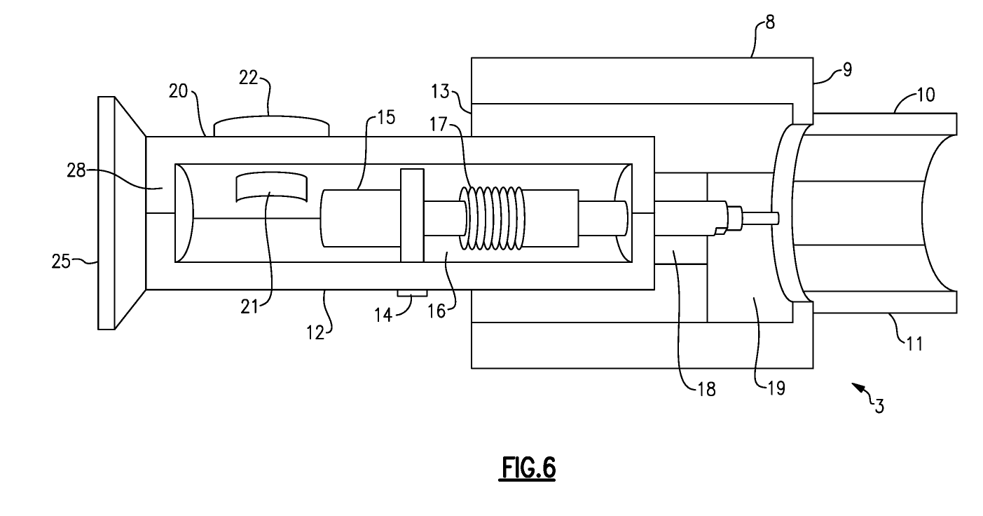

FIG. 5 illustrates the housing of another exemplary wearable aerosol inhaler 50 with a similar inhaler unit 3 to that of FIG. 1, highlighting an optional trap-door 23 that can slide out of the casing in order to expose the actuator 8 and canister 12 inside. Trap-door 23 can be secured with a friction fit or a similar fastener 24. The canister 12 can be refilled with the medicament, such as, for example, via a filling valve 20 rooted in the canister as shown in FIG. 6. The valve is typically a check-valve, that allows for only a one-way flow of the medicament. The inside of the exemplary valve is lined with a rubber ring 21 in which a valve stem can be inserted. A stopper 22 is shown in a default position plugging the valve and can be removed from the entrance to the valve for access. As understood by those skilled in the art, any suitable removable closure can be used in place of the exemplary stopper 22. Also, as understood by those skilled in the art, any suitable sealing device and method can be used in place of the exemplary rubber ring 21.

Within the canister as illustrated in more detail in FIG. 6, is a metered-dose valve 14 that dispenses a pre-determined volume of dosage. It includes a metering chamber 16 that fills with the correct amount of dosage, and a retaining cup 15 that holds the on-deck dose. A spring 17 is also incorporated into the exemplary mechanism and allows for the release of medicament through the valve stem 18 once pressure is applied to the valve stem, thus pushing back on the spring. Exemplary valve stem 18 is shown nested in one side of a nozzle 19, that is attached to the inside of the actuator 8. The other side of the nozzle faces out into the user's mouth and allows for medicament to be released in the form of aerosol droplets.

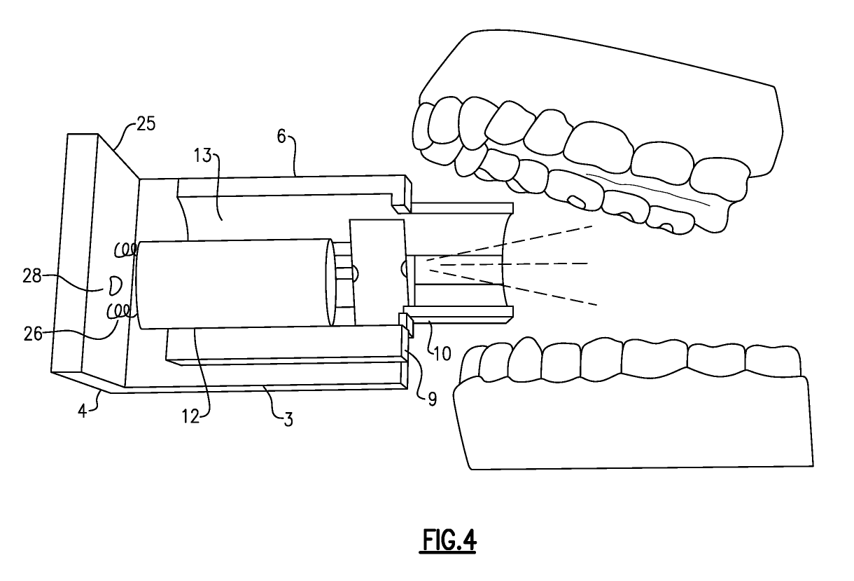

FIG. 4 illustrates the bite and push method involving the actuator 8, where the user pushes forward with their teeth placed on the bite pads 11 and mouth closed around the mouthpiece portion of the actuator 10. The actuator 8 then slides through the housing shell 6, until the canister 12 is pushing against a backboard 25, which is flush against the back of the outer casing 4. The actuator 8 is spring biased away from the backboard 25. The actuator pushes back on springs 26 located on either side of canister on backboard 25. With the proper amount of pressure, the valve stem 18 (FIG. 6) will be pushed fully into the nozzle 19, causing the spring 17 within metered-dose valve 14 to compress and release the medicament.

The method of use of a wristband inhaler typically includes: A) providing a wristband closed by at least one separation joint, the wristband flexible or hinged to open between a first end and a second end at the at least one separation joint, and an operable inhaler disposed within the first end, wherein in an open configuration, a partially exposed section and a mouthpiece are exposed to provide a dose of aerosol to a user, B) opening the wristband inhaler by pulling apart the first end and the second end, C) placing a user's lips about the partially exposed section, D) biting on the mouthpiece by the user, and E) pushing forward to release a pre-determined metered dose of an aerosol to the user.

In the exemplary embodiment of FIG. 4, as the canister 12 pushes back on the springboard 25, the dose counter 27 is set into motion by pressure on button 28, which is centered on the springboard 25. This timing corresponds with the moment the medication is released. Button 28 triggers dose counter mechanism 29 to turn the number roll 30. Number roll 30 displays the number of doses remaining through a clear window 31 in the side of the casing facing the user. Any suitable dose counter and dose counter advance device or system can be used.

In another embodiment of the wearable aerosol inhaler 1, the internal canister 12 can house only an amount of Albuterol which falls within FDA standards for dosage at a single time. For exemplary reference, each actuation of one traditional inhaler delivers 120 mcg of albuterol sulfate from the canister valve and 108 mcg of albuterol sulfate from the actuator mouthpiece (equivalent to 90 mcg of albuterol base). Such embodiments can forego the metered-dose valve 14, the filling valve 20, and the dose counter 27 along with their related components to allow for a compact, single-use device. The device itself or just the internal canister 12 can then be treated as a disposable element. In use, the user would follow identical actions as detailed above to release all of the aerosol (typically Albuterol) contained within internal canister 12 in a single spray, exhausting the supply of medication within the band.

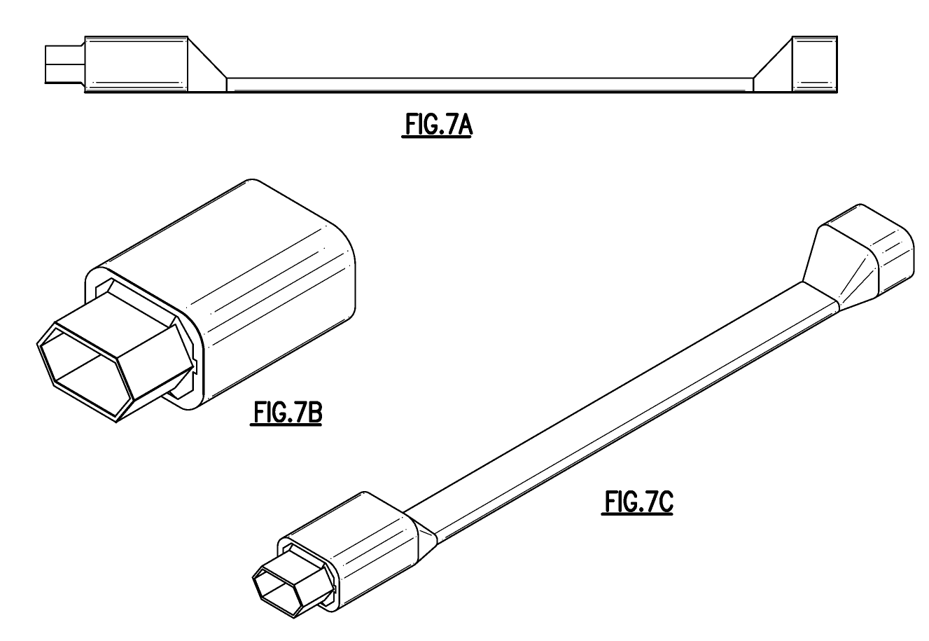

FIG. 7A shows a drawing of a side view of another exemplary embodiment of a wristband inhaler device. FIG. 7B shows an isometric view of a first end of the wristband inhaler device of FIG. 7A. FIG. 7C shows an isometric view of the wristband inhaler device of FIG. 7A.

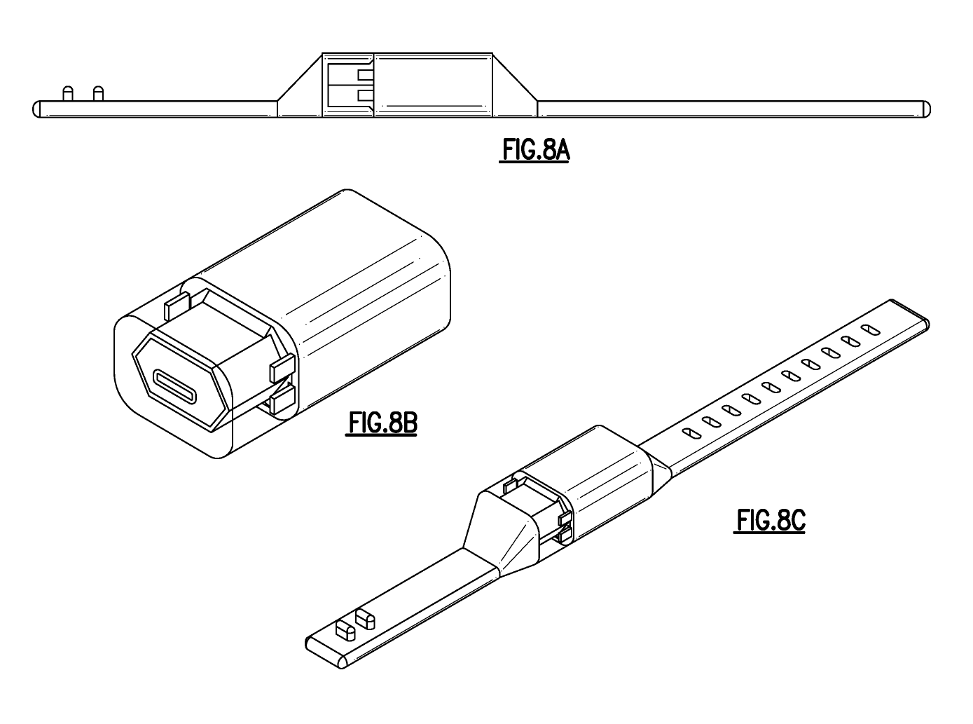

FIG. 8A shows a drawing of a side view of yet another exemplary embodiment of a wristband inhaler device with an adjustable strap. FIG. 8B shows a drawing an isometric view of a first end of the wristband inhaler device of FIG. 8A. FIG. 8C shows a drawing an isometric view of the wristband inhaler device of FIG. 8A. Any suitable adjustment mechanism can be used, such as, for example, any of the wrist strap adjustment devices known for use with wrist watches. For wrist watch type adjustable bands or straps, it is noted that the adjust and closure is typically about opposite to the first end having the inhaler disposed within and the second end, separable from the first end for inhaler use.

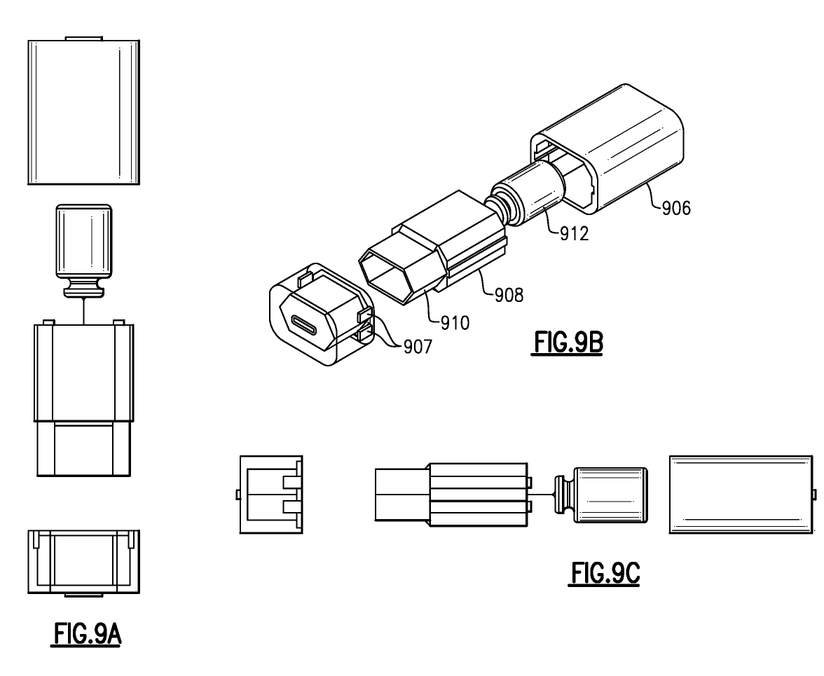

FIG. 9A shows a drawing of top view another exemplary first end. FIG. 9B shows a drawing of an isometric view of the first end of FIG. 9A. Magnets 907 enhance closure of the wristband inhaler between the first end and the second end at the separation joint. Mouthpiece 910 can be seen mechanically affixed to actuator 908. Reservoir 912 (e.g. a pod, replaceable or not, disposable, or not) can be seen to be housed in support base 906.

FIG. 9C shows a drawing of a side view of the first end of FIG. 9A.

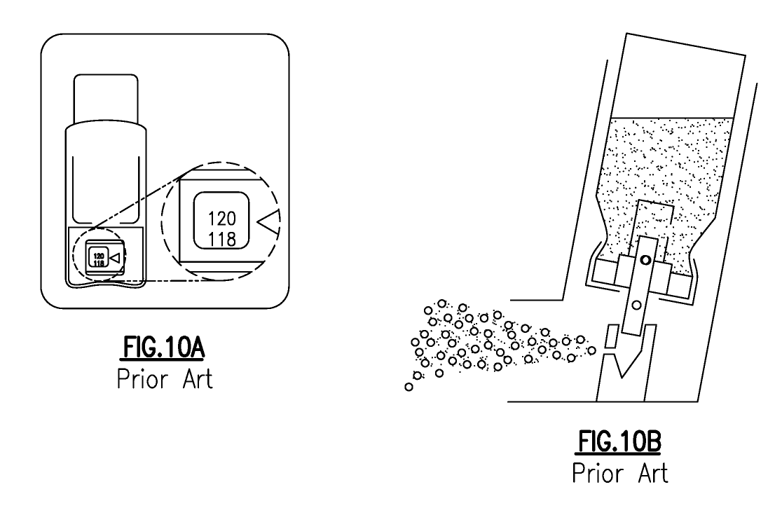

FIG. 10A shows a drawing of an exemplary mechanical dose counter of the prior art. FIG. 10B shows a drawing of an exemplary dose dispensing device of the prior art.

Any suitable means of wristband closer between at least a first end and a second end can be made by any suitable closure or locking structures. For example, there can be a ribbed or snap engagement (e.g. a ridge and groove snap engagement) such as including ridged members that are captured by corresponding troughs in the other part. There can also be closure by a friction fit or interference fit between structures of the first and second ends of the wristband.

Typically, the closure can be enhanced by magnets, such as rare-earth magnets, as described hereinabove. There can be corresponding magnets on either the first end and the second end, or there can be one or more magnets on one end and a corresponding ferrous member on the other end, or any combination thereof. For example, at least part of the mouthpiece can include a ferrous member. Or, there can be ferrous members or magnets on, in, or near any of the wristband ends or cavities, such as, for example, the cavity in the second end which can accept and provide a protective cover for any part of the inhaler, such as at least a portion of the mouth piece which can extend past the opened edge of the first end of the wristband.

There can also be additional seals, typically at the separation joint where the first end separates from the second end of the wristband, such as, for example, ring seals (e.g. “0” rings), overlapping shrouds, etc. to provide or to enhance weather resistance or water proofing.

Any suitable means of wristband separation and sizing can be used. There can be bands adjustable or not adjustable of various sizes, widths and lengths. There can also be a second detachable joint held similarly by magnets, where the wristband separates into two separate parts. In such embodiments, one of the detachable parts could be chosen to size the wristband. Also, such as in place of the wristband opening by flexible bending, there could be a structural hinge of any suitable hinge type about across from the opening joint, or at any other suitable position on the wristband.

The wearable aerosol inhaler as described hereinabove can be provided in a number of different configurations. In some embodiments, the wearable aerosol inhaler can be a single dose or a multidose inhaler. In some embodiments, whether single dose or a multidose, the wearable aerosol inhaler can be disposable or refillable and re-usable. Where the wearable aerosol inhaler is refillable and re-usable, there can be a refillable canister or a replaceable canister. For example, as shown in FIG. 9A to FIG. 9C, the canister can be a replaceable pod including a reservoir. Any suitable dispensing metered dose device which can be disposed, typically axially or co-axially in a first end of the wristband can be used.

While the wearable aerosol inhaler includes an inhaler disposed in the first end, the wearable aerosol inhaler can also be combined with any suitable conventional wrist worn device. For example, a wristband wearable aerosol inhaler could also include a conventional watch, a fitness tracker, and/or a GPS device. A wristband wearable aerosol inhaler including one or more additional conventional wrist worn devices can make the device more attractive and functional to encourage the device to be worn as often as possible so as to be more likely available when needed in an urgent situation or emergency.

In summary, Applicant's realized a wearable aerosol inhaler where rather than merely acting as a carry strap, the wristband itself is the inhaler. That is, the inhaler is integral and internally housed within at least one end of the wristband. Closure of the wearable aerosol inhaler wristband not only creates a wearable wristband, but also provides a protective cover for the end of the internal inhaler mechanisms, whether a single use inhaler, non-refillable multiple dose inhaler, or a refillable inhaler. Moreover, the bite and push forward dose operation means a dose can be had quickly and efficiently, especially in an emergency or panic situation, by merely opening the wristband. The bite and push dose operation solves the long felt need for an inhaler free of the problems of hand-to-mouth eye coordination associated with inhalers of the prior art.

It will be readily apparent to a person having ordinary skill in the art that many variations of the present invention can be made that still lie within the scope of this invention. Such variations include, but are not limited to, the specific embodiments described and shown in the figures and the above description. For example, the medicament reservoir and canister size may vary in dimensions and thus increase the number of doses the device is able to hold. The actuator size may also vary in proportion while still allowing a reasonable surface for which the user to interact with. Other materials such as Velcro or friction fit may be substituted for the magnetic closure as long as it ensures a strong bond that will not break unless directed actions are taken to open the band. The wristband or casing of the device may vary in material, as well as in ornamentation in order to accommodate various styles or indicate the purpose of the device or instructions for use.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A wearable inhaler comprising:
a wristband closed by at least one separation joint, said wristband flexible or hinged to open between a first end and a second end at said at least one separation joint; and
an operable inhaler disposed within said first end, wherein in an open configuration, a partially exposed section and a mouth piece are exposed to provide a metered dose of aerosol to a user.

2. The wearable inhaler of claim 1, wherein a longitudinal axis of said operable inhaler is aligned about axially or co-axially within said wristband.

3. The wearable inhaler of claim 1, wherein said mouth piece comprises at least one bite pad.

4. The wearable inhaler of claim 1, wherein said second end provides a protective cover for said operable inhaler disposed within said first end.

5. The wearable inhaler of claim 1, wherein said operable inhaler comprises a single dose inhaler.

6. The wearable inhaler of claim 1, wherein said operable inhaler comprises a multiple dose inhaler.

7. The wearable inhaler of claim 1, wherein said operable inhaler comprises a re-fillable inhaler.

8. The wearable inhaler of claim 7, wherein said operable inhaler comprises a trap refill door.

9. The wearable inhaler of claim 7, wherein said operable inhaler comprises a filling valve.

10. The wearable inhaler of claim 1, wherein said operable inhaler comprises an actuator slidably coupled to a housing shell.

11. The wearable inhaler of claim 10, wherein said operable inhaler comprises a backboard about flush against a back of an outer casing.

12. The wearable inhaler of claim 11, wherein said actuator is spring biased away from said backboard.

13. The wearable inhaler of claim 1, wherein said operable inhaler comprises a metered dose valve.

14. The wearable inhaler of claim 1, wherein said operable inhaler comprises a metering chamber to fill a predetermined dosage.

15. The wearable inhaler of claim 1, wherein said operable inhaler comprises a retaining cup to hold an on-deck dose.

16. The wearable inhaler of claim 1 wherein said operable inhaler comprises a dose counter.

17. The wearable inhaler of claim 1 wherein said operable inhaler comprises a dose button disposed in a backboard.

18. The wearable inhaler of claim 1 wherein said operable inhaler comprises an adjustable strap disposed between said first end and said second end.

19. The wearable inhaler of claim 18 wherein said wristband comprises a second separation point at one or more clasps or pins of said adjustable strap.

20. The wearable inhaler of claim 1 wherein said operable inhaler comprises a disposable or a replaceable pod.

21. The wearable inhaler of claim 1 further comprising a watch face or a fitness tracker to provide a physiological monitoring.

22. A method of use of a wristband inhaler comprising:
providing a wristband closed by at least one separation joint, said wristband flexible or hinged to open between a first end and a second end at said at least one separation joint, and an operable inhaler disposed within said first end, wherein in an open configuration, a partially exposed section and a mouthpiece are exposed to provide a dose of aerosol to a user;
opening said wristband inhaler by pulling apart said first end and said second end;
placing a user's lips about said partially exposed section;
biting on said mouthpiece by said user; and
pushing forward to release a metered dose of an aerosol to said user.
